(19) United States
(12) Patent Application Publication
(10) Pub. No.: US 2018/0271691 A1
(43) Pub. Date: Sep. 27, 2018

(54) ANKLE JOINT MECHANISM

(71) Applicant: HONDA MOTOR CO., LTD., Tokyo (JP)

(72) Inventor: Tomoya Amari, Wako-shi (JP)

(21) Appl. No.: 15/927,562

(22) Filed: Mar. 21, 2018

(30) Foreign Application Priority Data
Mar. 23, 2017 (JP) 2017-057804

(51) Int. Cl. A61F 5/01

(52) U.S. Cl. CPC A61F 5/0127; A61F 5/042; A61F 2005/0132

(57) ABSTRACT

An ankle joint mechanism to be fitted on an ankle of a user to assist motion of a body of the user, includes: a lower leg link to be disposed along a lower leg of the user; a foot holder configured to hold a foot of the user; a plantar/dorsi flexion mechanism configured to connect the lower leg link and the foot holder to each other so as to be pivotable about a plantar/dorsi flexion axis of an ankle joint of the user; an adduction/abduction mechanism configured to connect the lower leg link and the foot holder to each other so as to be pivotable about an adduction/abduction axis of the ankle joint; and an inversion/eversion mechanism configured to connect the lower leg link and the foot holder to each other so as to be pivotable about an inversion/eversion axis of the ankle joint.

### TECHNICAL FIELD

The present invention relates to an ankle joint mechanism fitted on an ankle of a user to assist motion of a body of the user.

### BACKGROUND ART

A walking assist device disclosed in JP2015-181661A includes a foot support on which a foot of the user is to be placed, a foot link connected to the foot support so as to be disposed on a lateral side of the foot, and a lower leg link configured to be disposed along a lower leg of the user. The foot link and the lower leg link are connected to each other through engagement of mutually facing uneven surfaces thereof and are fastened with each other by means of a screw. The uneven surfaces of the foot link and the lower leg link each have parallel ridges and grooves extending in the fore-and-aft direction, such that the uneven surfaces can engage each other at various vertical relative positions and their relative fore-and-aft positions can be adjusted by sliding along the ridges and grooves after the engagement. Thereby, the relative positions of the foot link and the lower leg link can be adjusted in the vertical and fore-and-aft directions before being fastened by the screw.

A human ankle joint includes a talocrural joint constituted of a tibia, a fibula, and a talus, and a subtalar joint constituted of the talus and a calcaneus. The talocrural joint mainly functions to allow plantar flexion/dorsiflexion motion (hereinafter referred to as “plantar/dorsi flexion”) of the ankle joint (also may be referred to as an ankle or a foot), while the subtalar joint mainly functions to allow pronation/supination motion (hereinafter referred to as “pronation/supination”) of the ankle joint. The axis of motion of the talocrural joint passes a point on the upper side of the talus, while the axis of motion of the subtalar joint passes a point on the lower side of the talus, whereby the plantar/dorsi flexion axis and the pronation/supination axis of the ankle joint are offset vertically from each other.

In the aforementioned conventional walking assist device (motion assist device), the foot support is connected to the foot link portion in such a manner that enables only plantar/dorsi flexion of the ankle joint, and therefore, when the ankle joint undergoes pronation/supination, the foot support cannot follow the motion, causing discomfort to the user.

Further, plantar/dorsi flexion of the ankle joint requires a large force to keep balance of the body, and therefore, with the conventional motion assist device, great discomfort tends to be caused to the user when pronation/supination takes place simultaneously with plantar/dorsi flexion.

### SUMMARY OF THE INVENTION

In view of such problems of the prior art, a primary object of the present invention is to provide an ankle joint mechanism that can reduce the discomfort caused to the user when the ankle joint of the user undergoes pronation/supination.

To achieve such an object, one aspect of the present invention provides an ankle joint mechanism (20) to be fitted on an ankle (1) of a user (U) to assist motion of a body of the user, comprising: a lower leg link (22) to be disposed along a lower leg (16) of the user; a foot holder (21) configured to hold a foot (8) of the user; a plantar/dorsi flexion mechanism (23) configured to connect the lower leg link and the foot holder to each other so as to be pivotable about a plantar/dorsi flexion axis (11) of an ankle joint (1) of the user; an adduction/abduction mechanism (30) configured to connect the lower leg link and the foot holder to each other so as to be pivotable about an adduction/abduction axis (13) of the ankle joint; and an inversion/eversion mechanism (46) configured to connect the lower leg link and the foot holder to each other so as to be pivotable about an inversion/eversion axis (14) of the ankle joint.

Here, the plantar/dorsi flexion axis (11) is defined as the axis of motion of the talocrural joint (5), the adduction/abduction axis (13) is defined as a vertical axis passing through a point of intersection between the pronation/supination axis (12) (the axis of motion of the subtalar joint (7)) and the transverse plane passing the subtalar joint, and the inversion/eversion axis (14) is defined as a line in the transverse plane obtained by projecting the pronation/supination axis onto the transverse plane. Namely, the adduction/abduction axis (13) is a rotation axis of the motion having only motion components of the pronation/supination in the transverse plane, and the inversion/eversion axis (14) is a rotation axis of the motion having motion components of the pronation/supination excluding those in the transverse plane.

According to this arrangement, the foot holder is connected to the lower leg link so as to be pivotable about each of the adduction/abduction axis and the inversion/eversion axis of the ankle joint, and thus, when the ankle joint undergoes pronation/supination, the foot holder is allowed to follow the motion of the ankle joint. Therefore, the discomfort caused to the user can be reduced. Further, the provision of the adduction/abduction mechanism and the inversion/eversion mechanism allows the foot holder to follow not only the motion about the pronation/supination axis but also complex motion resulting from combination of motions about multiple joints (the talocrural joint and the subtalar joint as well as Chopart joint and Lisfranc joint).

In the above arrangement, preferably, the inversion/eversion mechanism includes a first guide rail mechanism (46) configured to define an arc-shaped track having a center on the inversion/eversion axis (14).

According to this arrangement, the first guide rail mechanism can be positioned around the inversion/eversion axis (such as above the foot); namely, it is possible to avoid positioning the inversion/eversion mechanism including the first guide rail mechanism on the inversion/eversion axis in front of and/or at the back of the subtalar joint (or in front of and/or at the back of the foot). Therefore, motion of the user, such as walking, is not hindered.

In the above arrangement, the first guide rail mechanism (46) includes a first arc-shaped rail (43) connected to a side of the foot holder (21) to be disposed in front of a lower portion of the lower leg (16), and a first slider (42) connected to a side of the lower leg link (22) and configured to be slidable along the first arc-shaped rail.

According to this arrangement, the first arc-shaped rail (and hence the hoot holder) is allowed to swing about the inversion/eversion axis. Thus, the inversion/eversion mechanism can be realized with a simple structure. In addition, the foot holder can be supported by the first arc-shaped rail with high support stiffness.

In the above arrangement, preferably, the first arc-shaped rail (43) is connected to the foot holder (21) in a height adjustable manner.

The height from the sole to the inversion/eversion axis varies for different users. According to this arrangement, the distance from the foot placement surface of the foot holder to the center of the track of the first arc-shaped rail can be adjusted, such that the first arc-shaped rail is disposed at a position in conformity with the inversion/eversion axis of the user.

In the above arrangement, preferably, the plantar/dorsi flexion mechanism (23) includes an upper member (24) connected to a side of the lower leg link (22), and a lower member (32) connected to a side of the foot holder (21) and supported by the upper member so as to be pivotable about the plantar/dorsi flexion axis (11).

According to this arrangement, the upper member and the lower member can be easily disposed at positions where these members hardly interfere with the motion of the ankle joint and the leg. In addition, the plantar/dorsi flexion mechanism can be realized with a simple structure.

In the above arrangement, preferably, the lower leg link (22) is configured to be disposed in front of the lower leg (16), wherein the upper member (24) has a U-shape in plan view to surround a lower front portion of the lower leg and is connected at a central part thereof to the lower leg link, and the lower member (32) has a U-shape in plan view with two ends thereof being pivotably supported by corresponding ends of the upper member.

According to this arrangement, the lower leg link, the upper member, and the lower member can be disposed at positions above the foot where these members hardly interfere with the motion of the ankle joint and the leg, and in addition, stiffness of the upper member and the lower member is improved.

In the above arrangement, preferably, the adduction/abduction mechanism includes a second guide rail mechanism (30) configured to define an arc-shaped track having a center on the adduction/abduction axis (13).

According to this arrangement, the second guide rail mechanism can be positioned around the adduction/abduction axis (for example, in front of the lower portion of the lower leg); namely, it is possible to avoid positioning the adduction/abduction mechanism on the adduction/abduction axis. Thus, an adduction/abduction mechanism that does not hinder the motion of the user, such as walking, can be realized.

In the above arrangement, preferably, the second guide rail mechanism includes a second arc-shaped rail (25) connected to a side of the foot holder (21) to be disposed in front of a lower portion of the lower leg (16), and a second slider (29) connected to a side of the lower leg link (22) and configured to be slidable along the second arc-shaped rail.

According to this arrangement, the second arc-shaped rail is allowed to swing about the adduction/abduction axis, and therefore, the adduction/abduction mechanism can be realized with a simple structure. In addition, the foot holder can be supported by the second arc-shaped rail with high support stiffness.

In the above arrangement, preferably, the ankle joint mechanism further includes an adduction/abduction angle adjustment mechanism (40) configured to connect the lower leg link (22) and the foot holder (21) to each other such that an angle between the lower leg link and the foot holder about the adduction/abduction axis (13) can be adjusted in accordance with a neutral position of the foot about the adduction/abduction axis.

The neutral position of the foot about the adduction/abduction axis or the angle of the longitudinal axis of the foot in the neutral state (degree of toe-in/out) varies depending on users. However, according to the above arrangement, the neutral angle of the foot holder about the adduction/abduction axis can be adjusted for each user. This allows an appropriate tolerable adduction/abduction angle from the neutral position of the foot to be set for each of adduction and abduction of the ankle joint.

In the above arrangement, preferably, the adduction/abduction angle adjustment mechanism includes a third guide rail mechanism (40) configured to define an arc-shaped track having a center on the adduction/abduction axis (13).

According to this arrangement, third guide rail mechanism can be positioned around the adduction/abduction axis (for example, in front of a lower portion of the lower leg); namely, it is possible to avoid positioning the adduction/abduction angle adjustment mechanism on the adduction/abduction axis. Thus, an adduction/abduction angle adjustment mechanism that does not interfere with the motion of the user, such as walking, can be realized.

In the above arrangement, preferably, the third guide rail mechanism includes a third arc-shaped rail (33) connected to a side of the lower leg link (22) to be disposed in front of a lower portion of the lower leg (16), and a third slider (37) connected to a side of the foot holder (21) and configured to be slidable along the third arc-shaped rail.

According to this arrangement, the third slider is allowed to swing about the adduction/abduction axis with a simple structure.

### EFFECT OF THE INVENTION

Thus, according to an aspect of the present invention, there is provided an ankle joint mechanism that can reduce the discomfort caused to the user when the ankle joint of the user undergoes pronation/supination.

### BRIEF DESCRIPTION OF THE DRAWINGS

FIGS. 1A and 1B are schematic diagrams for explaining a human ankle joint, FIG. 1 showing a rear view of a right foot, and FIG. 1B showing a top view of the right foot;

FIGS. 2A and 2B are schematic diagrams for explaining a human ankle joint, FIG. 2A showing an inner side view of the right foot, and FIG. 2B showing a top view of the right foot;

FIG. 3 is a schematic diagram for explaining reference planes of a human foot;

FIG. 4 is a perspective view of an ankle joint mechanism according to an embodiment fitted on a foot of a user; and

FIG. 5 is a sectional view taken along line V-V in FIG. 4.

### DESCRIPTION OF THE PREFERRED EMBODIMENT(S)

In the following, preferred embodiments of the present invention will be described in detail with reference to the appended drawings.

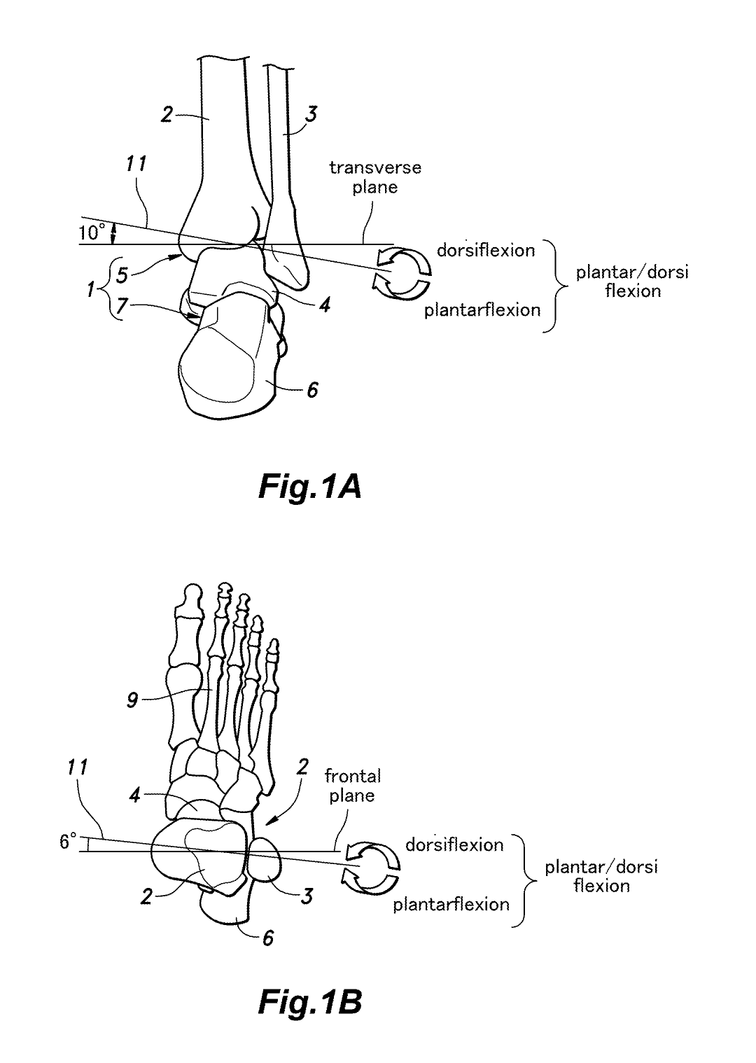

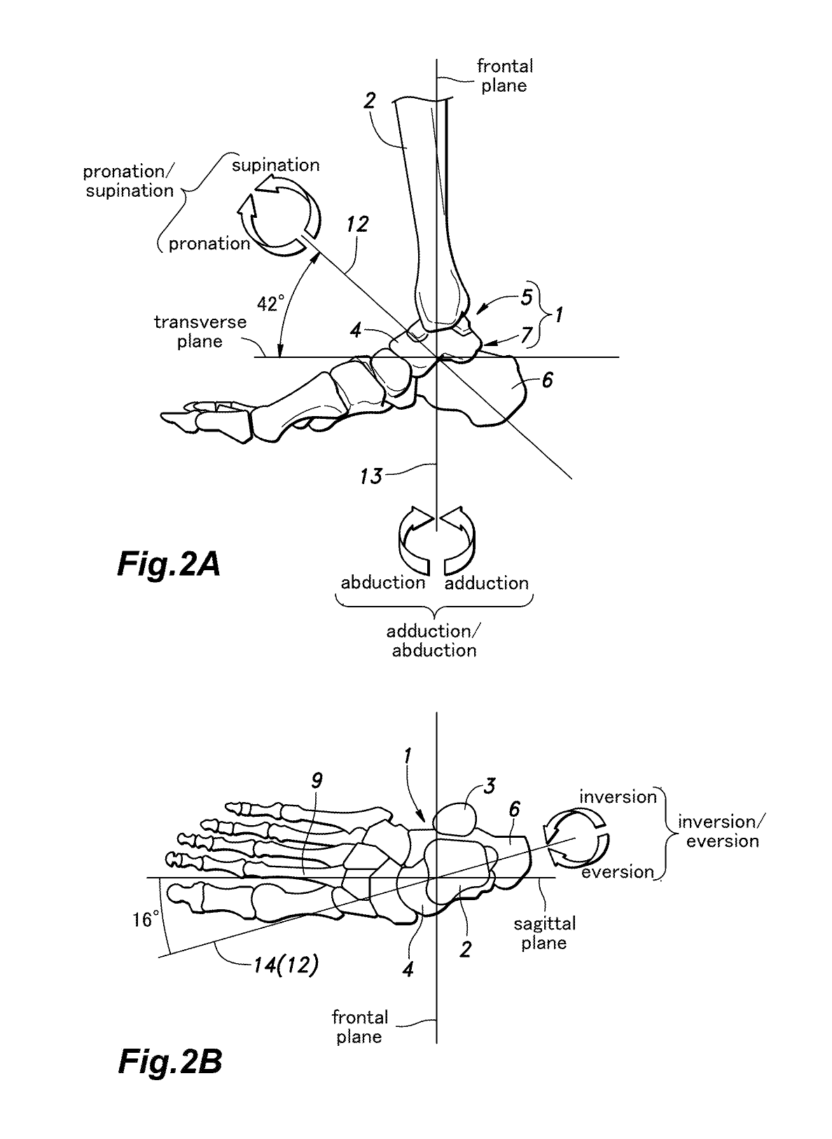

First, a human ankle joint 1 will be described with reference to FIGS. 1A, 1B, 2A, and 2B. FIG. 1A shows a rear view of a right foot, FIG. 1B shows a top view of the right foot, FIG. 2A shows an inner side view of the right foot, and FIG. 2B shows a top view of the right foot. The ankle joint 1 includes a talocrural joint 5 constituted of a tibia 2, a fibula 3, and a talus 4, and a subtalar joint 7 constituted of the talus 4 and a calcaneus 6. The talocrural joint 5 mainly functions to allow plantar/dorsi flexion of the ankle joint 1, while the subtalar joint 7 mainly functions to allow pronation/supination of the ankle joint 1.

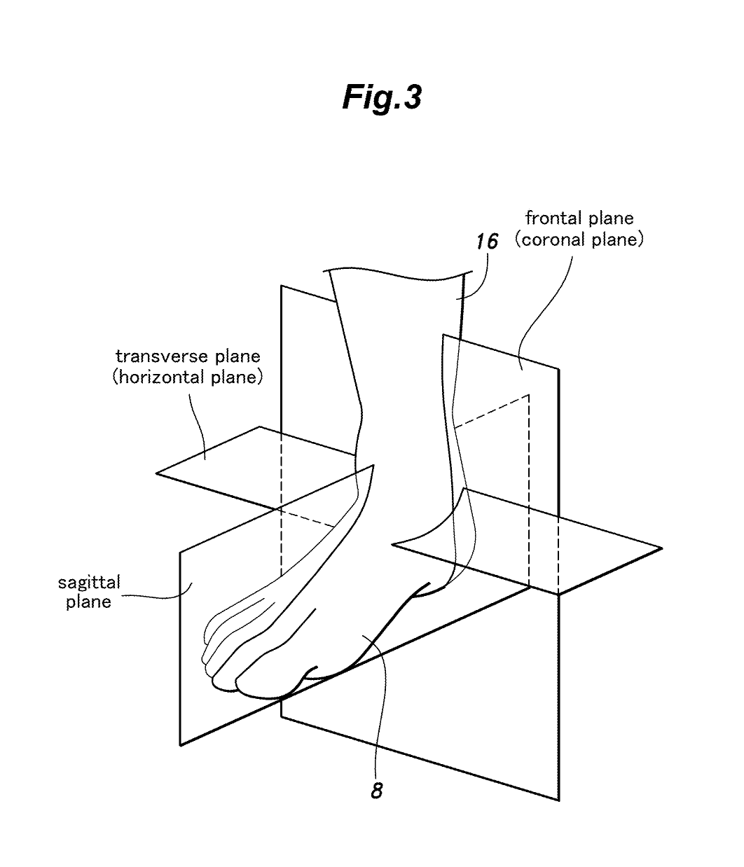

Here, with reference to FIG. 3, reference planes of a foot 8 are determined. In a basic limb position, which is a limb position in a stationary standing posture with the longitudinal axis of the foot 8 (longitudinal axis of the second metatarsal bone 9 (FIG. 2B)) being parallel to the longitudinal axis of the other foot, a transverse plane is defined as a plane parallel to the plantar surface (horizontal plane), a sagittal plane is defined as a plane perpendicular to the transverse plane and passing the longitudinal axis of the second metatarsal bone 9, and a frontal plane (coronal plane) is defined as a plane perpendicular to the transverse plane and the sagittal plane. A direction of extension of an intersection line between the transverse plane and the frontal plane will be referred to as a lateral direction or an inward-outward direction, a direction of extension of an intersection line between the transverse plane and the sagittal plane will be referred to as a fore-and-aft direction, and a direction of extension of an intersection line between the frontal plane and the sagittal plane will be referred to as an up-down direction or a vertical direction.

As shown in FIGS. 1A and 1B, the axis of motion 11 of the talocrural joint 5 extends to pass a point on the upper side of the talus 4 in a substantially lateral direction, though precisely, the axis of motion 11 of the talocrural joint 5 is inclined relative to the transverse plane by about 10 degrees in such a direction that the inner section thereof is positioned higher than the outer section as shown in FIG. 1A, and relative to the frontal plane by about 6 degrees in such a direction that the inner section thereof is positioned more forward than the outer section as shown in FIG. 1B. Therefore, plantar/dorsi flexion at the talocrural joint 5 results in motion primarily in the sagittal plane. On the other hand, as shown in FIGS. 2A and 2B, the axis of motion 12 of the subtalar joint 7 extends to pass a point on the lower side of the talus 4, and is inclined relative to the transverse plane by about 42 degrees in such a direction that the front section thereof is positioned higher than the rear section as shown in FIG. 2A, and relative to the sagittal plane by about 16 degrees in such a direction that the front section thereof is positioned more inward than the rear section as shown in FIG. 2B. Therefore, motion about the axis of motion 12 of the subtalar joint 7 results in movements in the frontal, transverse, and sagittal planes. Particularly, an initial motion from the basic limb position successively causes a movement in the frontal plane, in the transverse plane, and in the sagittal plane in this order.

With regard to the terminologies relating to the motion of the foot 8, such as “plantarflexion,” “dorsiflexion,” “abduction,” “adduction,” “inversion,” and “eversion,” in some documents, plantarflexion and dorsiflexion are defined as referring to the motion (motion component) in the sagittal plane, abduction and adduction are defined as referring to the motion in the transverse plane, and inversion and eversion are defined as referring to the motion in the frontal plane. However, in the present description, the following definitions are adopted. Namely, as shown in FIGS. 1A and 1B, a plantar/dorsi flexion axis is defined as the axis of motion 11 of the talocrural joint 5, and the motion about the plantar/dorsi flexion axis 11 is referred to as plantar/dorsi flexion, where plantar flexion refers to such motion in a direction to lower the toes and dorsiflexion refers to such motion in a direction to lift up the toes. Also, as shown in FIGS. 2A and 2B, a pronation/supination axis is defined as the axis of motion 12 of the subtalar joint 7, and the motion about the pronation/supination axis 12 is referred to as pronation/supination, where pronation refers to such motion in a direction to move the sole outward and supination refers to such motion in a direction to move the sole inward.

Further, an adduction/abduction axis 13 is defined as a vertical line passing through a point of intersection between the pronation/supination axis 12 and the transverse plane that passes the subtalar joint 7 (namely, the axis 13 being an intersection line between the frontal plane and the sagittal plane), and an inversion/eversion axis 14 is defined as a horizontal line in the transverse plane obtained by projecting the pronation/supination axis 12 onto the transverse plane (horizontal plane) passing the subtalar joint 7. Namely, the inversion/eversion axis 14 is a rotation axis of the motion having motion components of the pronation/supination (motion about the pronation/supination axis 12) excluding those in the transverse plane. The motion in the transverse plane about the adduction/abduction axis 13 is referred to as adduction/abduction (internal/external rotation), where adduction (internal rotation) refers to such motion in a direction to move the sole inward, and abduction (external rotation) refers to such motion in a direction to move the sole outward. Also, the motion about the inversion/eversion axis 14 (mainly consisting of motion in the frontal plane but also including motion in the sagittal plane) is referred to as inversion/eversion, where inversion refers to such motion in a direction to move the sole inward, and eversion refers to such motion in a direction to move the sole outward.

In the present description, pronation is regarded as a motion that combines eversion which primarily has motion components in the frontal plane and abduction which primarily has motion components in the transverse plane, and supination is regarded as a motion that combines inversion which primarily has motion components in the frontal plane and adduction which primarily has motion components in the transverse plane, where the motion components of pronation/supination in the sagittal plane are ignored as they are trivial. On the other hand, plantar/dorsi flexion in the present description is regarded as including all movements about the plantar/dorsi flexion axis 11 which may have motion components in the frontal and transverse planes in addition to motion components in the sagittal plane.

As described above, the ankle joint 1 may undergo not only plantar/dorsi flexion but also inversion/eversion and/or adduction/abduction, and the plantar/dorsi flexion axis 11 of the talocrural joint 5 relating to plantar/dorsi flexion and the pronation/supination axis 12 of the subtalar joint 7 relating to inversion/eversion and adduction/abduction are offset vertically from each other in the basic limb position. An ankle joint mechanism 20 according to an embodiment of the present invention is configured so as to be capable of following such motion of the foot 8. In the following, the ankle joint mechanism 20 will be described in detail.

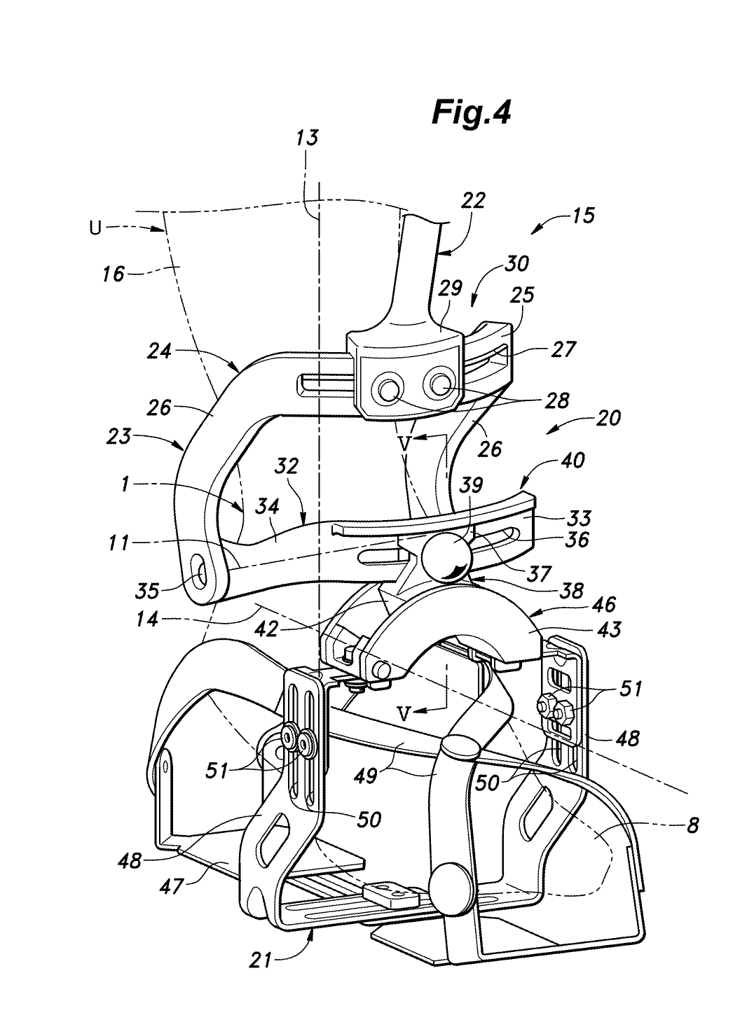

FIG. 4 is a perspective view of the ankle joint mechanism 20 according to the embodiment. The ankle joint mechanism 20 is a part of a motion assist device 15 for assisting motion of a body of a user U, and is fitted on the ankle joint (ankle) 1 of the user U. The motion assist device 15 may be configured to assist walking motion of the legs of the user U or to assist bending and stretching motion of the legs of the user U, for example. The motion assist device 15 is configured to contact the ground such that the user U does not have to bear the entire weight of the device. The part of the motion assist device 15 that contacts the ground is a foot holder 21 configured to hold the foot 8 of the user U placed on a foot placement surface thereof and to move together with the foot 8 following the motion of the leg of the user U. In the following, the structure of the ankle joint mechanism 20 will be described, assuming that the limb of the user U is in the basic limb position.

The ankle joint mechanism 20 includes a lower leg link 22 configured to be disposed in front of a lower leg 16 of the user U and to extend vertically along the lower leg 16. A lower end of the lower leg link 22 is connected to an upper member 24 constituting a hinge mechanism 23. The upper member 24 is formed by processing a metallic plate, such as that made of aluminum or the like, and includes a second arc-shaped rail 25 extending generally laterally in front of a lower portion of the lower leg 16 to exhibit an arc shape having a center on the adduction/abduction axis 13, which extends vertically behind the second arc-shaped rail 25, and a pair of left and right second arms 26 extending from the left and right ends of the second arc-shaped rail 25 obliquely rearward and downward on either side of the lower leg 16, whereby the upper member 24 has a U-shape in plan view to surround a lower front portion of the lower leg 16.

At a vertically middle part of the second arc-shaped rail 25 disposed in the central section of the upper member 24 is formed a second guide groove 27 (slot) extending in the lateral direction to exhibit an arc shape. A lower end part of the lower leg link 22 is configured to have front and rear walls defining a channel opening downward (or inverted U-shape) as seen in side sectional view and exhibits an arc shape conforming to the second arc-shaped rail 25. The second arc-shaped rail 25 is received in the channel defined in the lower end part of the lower leg link 22, with the front and rear walls of the lower end of the lower leg link 22 sandwiching the second arc-shaped rail 25 from front and rear, and is slidably engaged with the second arc-shaped rail 25 by means of two slide pins 28 passed through the second guide groove 27. Thus, the lower end part of the lower leg link 22 constitutes a second slider 29, and the second slider 29 and the second arc-shaped rail 25 constitute a second guide rail mechanism 30. The second arc-shaped rail 25, which is connected to the foot holder 21 via other members as described in the following, pivots about the adduction/abduction axis 13 of the ankle joint 1 by sliding relative to the second slider 29. Thus, the second guide rail mechanism 30 constitutes an adduction/abduction mechanism that connects the lower leg link 22 and the foot holder 21 to each other so as to be pivotable about the adduction/abduction axis 13 of the ankle joint 1.

The upper member 24 is connected to a lower member 32. The lower member 32 includes a third arc-shaped rail 33 extending generally laterally in front of the lower portion of the lower leg 16 to exhibit an arc shape having a center on the adduction/abduction axis 13, which extends vertically behind the third arc-shaped rail 33, and a pair of left and right third arms 34 extending from the left and right ends of the third arc-shaped rail 33 obliquely rearward and downward on either side of the lower leg 16, whereby the lower member has a U-shape in plan view to surround the lower front portion of the lower leg 16. The rear ends of the third arms 34 of the lower member 32 are pivotably supported by corresponding rear ends of the second arms 26 of the upper member 24 via respective pivots 35 provided to parts of the second arms 26 on the plantar/dorsi flexion axis 11. The lower member 32, which is connected to the foot holder 21 via other members as described in the following, pivots about the plantar/dorsi flexion axis 11 by pivoting relative to the upper member 24. Thus, the upper member 24 and the lower member 32 connected to the upper member 24 via the pivots 35 constitute the hinge mechanism 23 embodying a plantar/dorsi flexion mechanism that connects the lower leg link 22 and the foot holder 21 to each other so as to be pivotable about the plantar/dorsi flexion axis 11 of the ankle joint 1.

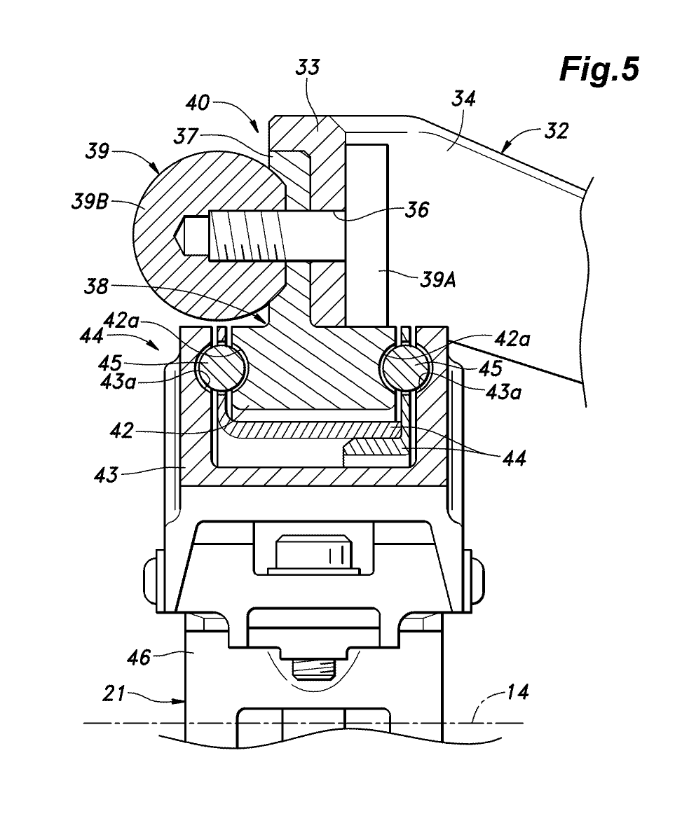

FIG. 5 is a sectional view taken along line V-V in FIG. 4. As shown in FIGS. 4 and 5, at a vertically middle part of the third arc-shaped rail 33 disposed at a central section of the lower member 32 is formed a third guide groove 36 (slot) extending in the lateral direction to exhibit an arc shape. This third guide groove 36 slidably engages with a slide member 38 including a third slider 37 which exhibits an arc shape conforming to the third arc-shaped rail 33. The third slider 37 is provided with a fixing member 39 which includes a bolt 39A and a spherical nut 39B for fixing the third slider 37 to the third arc-shaped rail 33 by fastening. The shaft of the bolt 39A serves as a slide pin, and when the fixing member 39 is loosened, the third slider 37 is slidably engaged with the third arc-shaped rail 33 via the shaft. Thereby, the third slider 37, the fixing member 39 and the third arc-shaped rail 33 constitute a third guide rail mechanism 40. The third slider 37, which is connected to the foot holder 21 via other members as described in the following, pivots about the adduction/abduction axis 13 by sliding along the third arc-shaped rail 33. Namely, the third guide rail mechanism 40 embodies an adduction/abduction angle adjustment mechanism that connects the lower leg link 22 and the foot holder 21 to each other such that an angle between the lower leg link 22 and the foot holder 21 about the adduction/abduction axis 13 can be adjusted in accordance with a neutral position of the foot 8 of the user U about the adduction/abduction axis 13.

The slide member 38 includes, in addition to the third slider 37, a first slider 42 depending from the third slider 37 (thus, the first slider 42 is connected to the lower leg link 22 via the third slider 37, third arc-shaped rail 33 (lower member 32), pivots 35, upper member 24 (second arc-shaped rail 25), and second slider 29 (lower end part of the lower leg link 22)). Via the first slider 42, the slide member 38 slidably engages with a first arc-shaped rail 43 exhibiting an arc shape having a center on the inversion/eversion axis 14, which extends substantially in the fore-and-aft direction below the first arc-shaped rail 43. Specifically, the first arc-shaped rail 43 is configured to have front and rear walls defining a channel-shaped cross section opening upward, and a pair of arc-shaped guide grooves 43a each having an arc-shaped cross section are respectively formed on opposing inner surfaces of the front and rear walls to extend along the arc shape of the arc-shaped rail 43. Front and rear surfaces of the first slider 42 are formed with corresponding arc-shaped guide grooves 42a having a shape corresponding to that of the arc-shaped guide grooves 43a such that the corresponding arc-shaped guide grooves 42a oppose the arc-shaped guide grooves 43a, respectively. A plurality of bearing balls 45 retained in a retainer 44 are provided between each opposing pair of the arc-shaped guide groove 43a and the corresponding arc-shaped guide groove 42a, such that the first slider 42 slides along the first arc-shaped rail 43 via the bearing balls 45. Thereby, the first slider 42 and the first arc-shaped rail 43 constitute a first guide rail mechanism 46. The first arc-shaped rail 43, which is connected to the foot holder 21 as described in the following, pivots about the inversion/eversion axis 14 of the ankle joint 1 by sliding relative to the first slider 42, which is connected to the lower leg link 22 as described above. Namely, the first guide rail mechanism 46 embodies an inversion/eversion mechanism that connects the lower leg link 22 and the foot holder 21 to each other so as to be pivotable about the inversion/eversion axis 14 of the ankle joint 1.

As shown in FIG. 4, the first arc-shaped rail 43 is disposed in front of the lower portion of the lower leg 16 and above the foot 8, extends generally in the lateral direction, and has lateral ends connected to corresponding lateral side portions of the foot holder 21. The foot holder 21 includes a bottom plate 47 having a foot placement surface, left and right side plates 48 extending upward from the left and right sides of the bottom plate 47 at a longitudinally intermediate part of the bottom plate 47, and a band 49 made of an elastic material, such as rubber, and detachably attached to the bottom plate 47 to hold the foot 8 of the user U placed on the bottom plate 47, whereby the foot holder 21 is configured to be capable of holding the foot 8 of the user U. Each of the side plates 48 is formed with two rows of slots 50 extending vertically, and the first arc-shaped rail 43 is connected to the foot holder 21 in a height adjustable manner by fastening members 51, such as bolts and nuts, passed through the slots 50.

The ankle joint mechanism 20 configured as described above operates in accordance with motion of the ankle joint 1 of the user U, as follows. When the ankle joint 1 of the user U undergoes plantar/dorsi flexion, the lower member 32 of the hinge mechanism 23 pivots about the plantar/dorsi flexion axis 11 owing to the pivots 35 provided on the plantar/dorsi flexion axis 11. When the ankle joint 1 of the user U undergoes pronation/supination, the first arc-shaped rail 43 of the inversion/eversion mechanism pivots relative to the first slider 42 of the slide member 38 about the inversion/eversion axis 14, and simultaneously, the second arc-shaped rail 25 (the upper member 24) of the adduction/abduction mechanism pivots relative to the second slider 29 about the adduction/abduction axis 13. When the ankle joint 1 of the user U undergoes plantar/dorsi flexion and pronation/supination simultaneously, the above actions are performed simultaneously.

In the following, technical advantages of the ankle joint mechanism 20 having the above structure will be described.

The ankle joint mechanism 20 is provided with an adduction/abduction mechanism which is embodied by the second guide rail mechanism 30 that connects the lower leg link 22 and the foot holder 21 to each other so as to be pivotable about the adduction/abduction axis 13, and an inversion/eversion mechanism embodied by the first guide rail mechanism 46 that connects the lower leg link 22 and the foot holder 21 to each other so as to be pivotable about the inversion/eversion axis 14, whereby when the ankle joint 1 undergoes pronation/supination, the foot holder 21 is allowed to follow the motion of the ankle joint 1. Therefore, the discomfort caused to the user U is reduced. Further, the foot holder 21 can follow not only the motion about the pronation/supination axis 12 but also complex motion resulting from combination of motions about multiple joints (such as the talocrural joint 5 and the subtalar joint 7 as well as Chopart joint and Lisfranc joint).

The inversion/eversion mechanism is embodied by the first guide rail mechanism 46 configured to define an arc-shaped track having a center on the inversion/eversion axis 14, and such a structure allows the first guide rail mechanism 46 to be positioned around the inversion/eversion axis 14, such as above the foot 8. Namely, it is possible to avoid positioning the inversion/eversion mechanism on the inversion/eversion axis 14 in front of and/or at the back of the subtalar joint 7 (or in front of and/or at the back of the foot 8). Therefore, the inversion/eversion mechanism does not hinder motion of the user U, such as walking.

The first guide rail mechanism 46 is constituted by the first arc-shaped rail 43 connected to a side of the foot holder 21 to be disposed in front of the lower portion of the lower leg 16, and the first slider 42 connected to a side of the lower leg link 22 and configured to be slidable along the first arc-shaped rail 43, whereby the first arc-shaped rail 43 (and hence the foot holder 21) is allowed to swing about the inversion/eversion axis 14, and therefore, the inversion/eversion mechanism is realized with a simple structure. In addition, the foot holder 21 can be supported by the first arc-shaped rail 43 with high support stiffness.

The first arc-shaped rail 43 is connected to the foot holder 21 in a height adjustable manner, and this allows the distance from the foot placement surface of the foot holder 21 to the center of the track of the first arc-shaped rail 43 to be adjusted. Thereby, the first arc-shaped rail 43 can be disposed at a position in conformity with the inversion/eversion axis 14 of the user U. Therefore, the ankle joint mechanism 20 can be used commonly by various users having different distances from the sole to the inversion/eversion axis 14.

The hinge mechanism 23 serving as the plantar/dorsi flexion mechanism is constituted by the upper member 24 connected to a side of the lower leg link 22, and the lower member 32 connected to a side of the foot holder 21 and supported by the upper member 24 so as to be pivotable about the plantar/dorsi flexion axis 11. Thereby, the upper member 24 and the lower member 32 can be disposed at positions where these members hardly interfere with the motion of the ankle joint 1 and the leg, and in addition, the plantar/dorsi flexion mechanism can be realized with a simple structure.

In the illustrated embodiment, the lower leg link 22 is configured to be disposed in front of the lower leg 16, the upper member 24 has a U-shape in plan view to surround the lower front portion of the lower leg 16 and is connected at a central part thereof to the lower leg link 22, and the lower member 32 has a U-shape in plan view with two ends thereof being pivotably supported by corresponding ends of the upper member 24. Owing to this arrangement, the lower leg link 22, the upper member 24, and the lower member 32 can be disposed at positions above the foot 8 where these members hardly interfere with the motion of the ankle joint 1 and the leg, and in addition, stiffness of the upper member 24 and the lower member 32 is improved.

The adduction/abduction mechanism is embodied by the second guide rail mechanism 30 configured to define an arc-shaped track having a center on the adduction/abduction axis 13, and such a second guide rail mechanism 30 can be positioned around the adduction/abduction axis 13, such as in front of the lower portion of the lower leg 16. Namely, it is possible to avoid positioning the adduction/abduction mechanism on the adduction/abduction axis 13. Thus, an adduction/abduction mechanism that does not hinder the motion of the user U, such as walking, can be realized.

The second guide rail mechanism 30 is constituted by the second arc-shaped rail 25 connected to a side of the foot holder 21 to be disposed in front of the lower portion of the lower leg 16, and the second slider 29 connected to a side of the lower leg link 22 and configured to be slidable along the second arc-shaped rail 25, whereby the second arc-shaped rail 25 (and hence the foot holder 21) is allowed to swing about the adduction/abduction axis 13, and therefore, the adduction/abduction mechanism is realized with a simple structure. In addition, the foot holder 21 can be supported by the second arc-shaped rail 25 with high support stiffness.

In the illustrated embodiment, the ankle joint mechanism 20 further includes an adduction/abduction angle adjustment mechanism embodied by the third guide rail mechanism 40 that connects the lower leg link 22 and the foot holder 21 to each other such that an angle between the lower leg link 22 and the foot holder 21 about the adduction/abduction axis 13 can be adjusted in accordance with a neutral position of the foot 8 about the adduction/abduction axis 13. Thereby, the neutral angle (zero angle) of the foot holder 21 about the adduction/abduction axis 13 can be adjusted for each user U. This allows an appropriate tolerable adduction/abduction angle from the neutral position of the foot 8 to be set for each of adduction and abduction of the ankle joint 1, and thereby allows the ankle joint mechanism 20 to be commonly used by various users having different neutral positions of the foot 8 about the adduction/abduction axis 13 or having different angles of the longitudinal axis of the foot 8 (degree of toe-in/out) in the neutral state.

The adduction/abduction angle adjustment mechanism includes the third guide rail mechanism 40 configured to define an arc-shaped track having a center on the adduction/abduction axis 13, whereby the third guide rail mechanism 40 can be disposed around the adduction/abduction axis 13, such as in front of the lower portion of the lower leg 16. Namely, it is possible to avoid positioning the adduction/abduction angle adjustment mechanism on the adduction/abduction axis 13, and therefore, an adduction/abduction angle adjustment mechanism that does not hinder the motion of the user U, such as walking, can be realized.

The third guide rail mechanism 40 is constituted by the third arc-shaped rail 33 connected to a side of the lower leg link 22 to be disposed in front of the lower portion of the lower leg 16, and the third slider 37 connected to a side of the foot holder 21 and configured to be slidable along the third arc-shaped rail 33, whereby the third slider 37 is configured to be swingable about the adduction/abduction axis 13.

A preferred embodiment of the present invention has been described in the foregoing, but the present invention is not limited to the above-described embodiment and various modifications and alterations are possible. For example, in the above embodiment, the second guide rail mechanism 30 constituting the adduction/abduction mechanism is provided between the lower leg link 22 and the hinge mechanism 23 constituting the plantar/dorsi flexion mechanism, and the third guide rail mechanism 40 constituting the adduction/abduction angle adjustment mechanism is provided between the hinge mechanism 23 and the first guide rail mechanism 46 constituting the inversion/eversion mechanism. However, it is also possible to provide the slide pins 28 to the third guide rail mechanism 40 to make the third guide rail mechanism 40 function as the adduction/abduction mechanism while providing the fixing member 39 to the second guide rail mechanism 30 to make the second guide rail mechanism 30 function as the adduction/abduction angle adjustment mechanism. Besides, the concrete structure, number, angle, etc. of the structural elements of the present invention shown in the above embodiment may be changed appropriately within the scope of the present invention defined by the claims. Further, not all of the structural elements shown in the above embodiment are necessarily indispensable, and they may be selectively adopted as appropriate.

1. An ankle joint mechanism to be fitted on an ankle of a user to assist motion of a body of the user, comprising:
a lower leg link to be disposed along a lower leg of the user;
a foot holder configured to hold a foot of the user;
a plantar/dorsi flexion mechanism configured to connect the lower leg link and the foot holder to each other so as to be pivotable about a plantar/dorsi flexion axis of an ankle joint of the user;
an adduction/abduction mechanism configured to connect the lower leg link and the foot holder to each other so as to be pivotable about an adduction/abduction axis of the ankle joint; and
an inversion/eversion mechanism configured to connect the lower leg link and the foot holder to each other so as to be pivotable about an inversion/eversion axis of the ankle joint.

2. The ankle joint mechanism according to claim 1, wherein the inversion/eversion mechanism includes a first guide rail mechanism configured to define an arc-shaped track having a center on the inversion/eversion axis.

3. The ankle joint mechanism according to claim 2, wherein the first guide rail mechanism includes a first arc-shaped rail connected to a side of the foot holder to be disposed in front of a lower portion of the lower leg, and a first slider connected to a side of the lower leg link and configured to be slidable along the first arc-shaped rail.

4. The ankle joint mechanism according to claim 3, wherein the first arc-shaped rail is connected to the foot holder in a height adjustable manner.

5. The ankle joint mechanism according to claim 1, wherein the plantar/dorsi flexion mechanism includes an upper member connected to a side of the lower leg link, and a lower member connected to a side of the foot holder and supported by the upper member so as to be pivotable about the plantar/dorsi flexion axis.

6. The ankle joint mechanism according to claim 5, wherein the lower leg link is configured to be disposed in front of the lower leg, and
wherein the upper member has a U-shape in plan view to surround a lower front portion of the lower leg and is connected at a central part thereof to the lower leg link, and the lower member has a U-shape in plan view with two ends thereof being pivotably supported by corresponding ends of the upper member.

7. The ankle joint mechanism according to claim 1, wherein the adduction/abduction mechanism includes a second guide rail mechanism configured to define an arc-shaped track having a center on the adduction/abduction axis.

8. The ankle joint mechanism according to claim 7, wherein the second guide rail mechanism includes a second arc-shaped rail connected to a side of the foot holder to be disposed in front of a lower portion of the lower leg, and a second slider connected to a side of the lower leg link and configured to be slidable along the second arc-shaped rail.

9. The ankle joint mechanism according to claim 1, further comprising an adduction/abduction angle adjustment mechanism configured to connect the lower leg link and the foot holder to each other such that an angle between the lower leg link and the foot holder about the adduction/abduction axis can be adjusted in accordance with a neutral position of the foot about the adduction/abduction axis.

10. The ankle joint mechanism according to claim 9, wherein the adduction/abduction angle adjustment mechanism includes a third guide rail mechanism configured to define an arc-shaped track having a center on the adduction/abduction axis.

11. The ankle joint mechanism according to claim 10, wherein the third guide rail mechanism includes a third arc-shaped rail connected to a side of the lower leg link to be disposed in front of a lower portion of the lower leg, and a third slider connected to a side of the foot holder and configured to be slidable along the third arc-shaped rail.
